(19) United States
(12) Patent Application Publication
(10) Pub. No.: US 2005/0277918 A1
(43) Pub. Date: Dec. 15, 2005

(54) Electrosurgical cannula

(75) Inventors: Krishan Shah, Mississauga (CA); Subashini Chandran, Toronto (CA); Neil Godara, Mississauga (CA)

Correspondence Address: OGILVY RENAULT LLP, 1981 MCGILL COLLEGE AVENUE, SUITE 1600, MONTREAL, QC, H3A2Y3, CA

(73) Assignee: Baylis Medical Company Inc., Montreal (CA)

(21) Appl. No.: 11/207,707

(22) Filed: Aug. 22, 2005

Related U.S. Application Data
(63) Continuation-in-part of application No. 11/079,318, filed on Mar. 15, 2005.
Application No. 11/079,318 is a continuation-in-part of application No. 10/382,836, filed on Mar. 7, 2003.
(63) Continuation-in-part of application No. 11/125,247, filed on May 10, 2005.
Application No. 11/125,247 is a continuation-in-part of application No. 10/853,126, filed on May 26, 2004.

(51) Int. Cl. A61B 18/18; A61F 7/00; A61F 7/12

(52) U.S. Cl. 606/41; 607/99

(57) ABSTRACT

This invention discloses a cannula comprising an elongate shaft comprising a distal region and a proximal region and defining a lumen therebetween, and further comprising a wall defining at least one lateral aperture therethrough and a distal end defining at least one distal aperture. The distal region comprises an electrically exposed and conductive distal tip and the outer surface of the cannula between the distal tip and the proximal region is non-conductive.

### CROSS-REFERENCE TO RELATED APPLICATIONS

This application is a continuation-in-part of U.S. patent application Ser. No. 11/079,318, filed on Mar. 15, 2005, which is a continuation-in-part of U.S. patent application Ser. No. 10/382,836, filed on Mar. 7, 2003; and a continuation-in-part of U.S. patent application Ser. No. 11/125,247, filed on May 10, 2005, which is a continuation-in-part of U.S. patent application Ser. No. 10/853,126, filed on May 26, 2004. The disclosures of each of these applications are incorporated herein by reference.

### BRIEF DESCRIPTION OF THE DRAWINGS

In order that the invention may be readily understood, embodiments of the invention are illustrated by way of examples in the accompanying drawings, in which:

FIG. 1A is a top plan view of one embodiment of a cannula of the present invention;

FIG. 1B is a side sectional view along line 1B, of the cannula depicted in FIG. 1A;

FIGS. 2A-2C show top plan views, fragmented, of various embodiments of a cannula of the present invention;

FIG. 3A is a top plan view of a further embodiment of a cannula of the present invention;

FIG. 3B is a side sectional view, along line 3B, of the cannula depicted in FIG. 3A;

FIG. 4A is a top plan view of an additional embodiment of a cannula of the present invention;

FIG. 4B is a top plan view of one embodiment of a stylet;

FIG. 5A is a rear perspective view (from above) of an alternate embodiment of a stylet;

FIG. 5B is a rear perspective view (from above) of an embodiment of a cannula and a stylet; and

FIG. 6 is a schematic illustration of an embodiment of a system of the present invention.

### DETAILED DESCRIPTION OF THE INVENTION

With specific reference now to the drawings in detail, it is stressed that the particulars shown are by way of example and for purposes of illustrative discussion of embodiments of the present invention only, and are presented in the cause of providing what is believed to be the most useful and readily understood description of the principles and conceptual aspects of the invention.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Prior to describing the drawings in detail, it should be noted that, throughout this description and corresponding drawings, like numerals are used to refer to like elements of the present invention. Furthermore, as used herein, the singular forms “a”, “an”, and “the” refer to both the singular as well as plural, unless the context clearly indicates otherwise. For example, the term “a lumen” includes single or multiple lumens and can be considered equivalent to the phrase “at least one lumen”. Furthermore, for the purposes of this description, proximal indicates next to or nearer to the user, and distal indicates further away from the user.

Apparatus/System

Structure

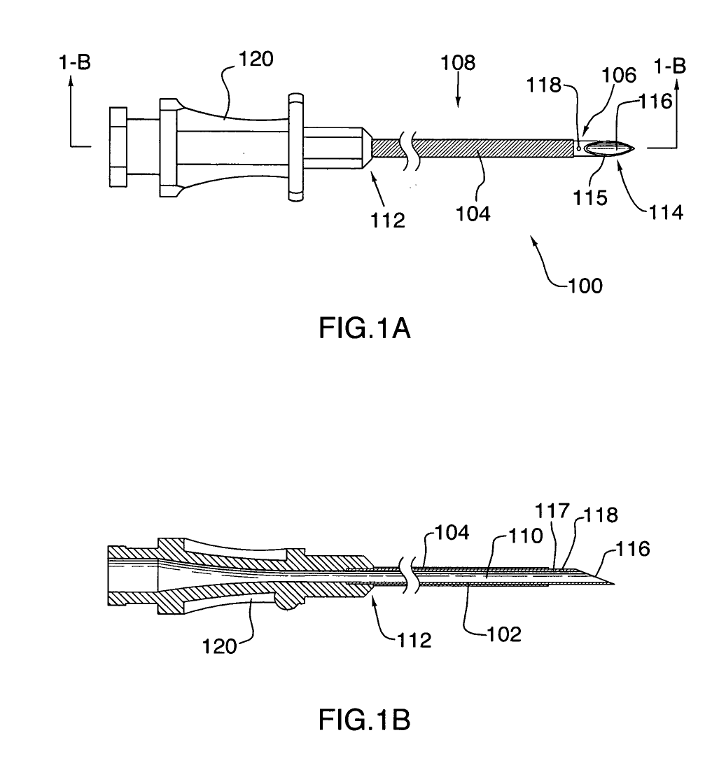

FIGS. 1A and 1B show a first embodiment of a cannula 100 of the present invention. FIG. 1A shows a top plan view of cannula 100 while FIG. 1B illustrates a sectional view of cannula 100, taken along the line 1B in FIG. 1A. In this embodiment, cannula 100 may be manufactured from an electrically conductive material, and an elongate shaft 102 of cannula 100 may be at least partially coated with an electrically insulating material 104. The region of cannula 100 comprising insulating material 104 may be referred to as insulated region 108. Alternatively, elongate shaft 102 may be made from a non-conductive material. Elongate shaft 102 comprises a proximal region 112 and a distal region 114. Distal region 114 comprises a distal tip 106 which may be electrically exposed and conductive. Thus, the outer surface of cannula 100, between distal tip 106 and proximal region 112, may be non-conductive. Due to the conductive nature of distal tip 106, and for the purposes of describing some embodiments of the present invention, distal tip 106 may be described as an active electrode or active tip 106. One or more markers may be present on cannula 100 to aid in visualization during insertion and/or treatment, for example, a radiopaque marker located at the boundary between insulating material 104 and active tip 106 may allow active tip 106 to be readily distinguishable from insulated region 108 using radiographic imaging. Distal tip 106 may be sharp to facilitate penetration into a patient's body. Alternatively, distal tip 106 may be blunt, rounded, straight, beveled, rigidly bent, or may take on other forms depending on the particular application.

Shaft 102 defines a lumen 110 extending longitudinally between proximal region 112 and distal region 114 of cannula 100. In this first embodiment, a distal end 115 of shaft 102 defines at least one distal aperture 116 and a wall 117 of shaft 102 defines at least one lateral aperture 118 therethrough. In this embodiment, distal aperture 116 and lateral aperture 118 are both in communication with lumen 110 of shaft 102. For the purposes of this disclosure, the term ‘lateral aperture’ is defined as an aperture, opening or hole defined through a lateral or radial surface, for example a wall, of cannula 100.

In some embodiments, cannula 100 further comprises a hub 120 associated with proximal region 112. Hub 120 may be manufactured from ABS (Acrylonitrile Butadiene Styrene) or other materials and may be attached to shaft 102 using various methods, including but not limited to insert molding, gluing and other forms of bonding. In the context of the instant disclosure, the term hub indicates a fitting or any other means of facilitating a secure connection between separate components such as a cannula and a probe. As such, hub 120 may be structured to cooperatively engage and mate with a probe, stylet or other device which may be introduced into shaft 102. Hub 120 may also be useful as a handle to grasp and manipulate cannula 100. In those embodiments that comprise a hub, lumen 110 may be sized to simultaneously accommodate a stylet, probe or other device as well as a diagnostic or therapeutic agent. In other embodiments, lumen 110 may be designed to receive a probe, stylet or other device without having sufficient space to accommodate a diagnostic or therapeutic agent. In such embodiments, the probe, stylet or other device may be removed from lumen 110, allowing for injection of a diagnostic or therapeutic agent if so desired.

A cannula 100 as shown in the embodiment of FIG. 1 may allow for distribution of a treatment composition, such as diagnostic or therapeutic fluid agents through both lateral aperture 118 and distal aperture 116. Lateral aperture 118 may facilitate delivery to a larger volume of tissue, and may also induce the fluid agents to flow along the length of distal tip 106, parallel to cannula 100, which may effectively concentrate the diagnostic or therapeutic agents in an area to be treated. In other words, a treatment composition flowing through lateral aperture 118 may flow longitudinally along a surface of cannula 100 or may radiate away from cannula 100, which may not be possible using only a distal aperture 116. In this way, anesthetic, contrast fluids, or other diagnostic or therapeutic agents injected through hub 120 may be discharged through apertures 116 and 118 on a distal and lateral portion of cannula 100. Lateral aperture 118 may have a smooth or rounded wall, which may serve to minimise or reduce trauma to bodily tissue. For example, some embodiments may have a lateral aperture 118 with a smooth outer circumferential edge.

A distal aperture 116 may, alternatively or in addition, be used for the introduction or exposure of one or more treatment devices to the tissue or other material in the vicinity of cannula 100. Some treatment applications call for the use of measuring devices that directly contact the tissue being measured, in order to gain a more accurate reading. Such measuring devices, described in further detail below, may include, but are not limited to, temperature, impedance, or pressure sensors. The presence of a distal aperture 116 allows a measuring device to be inserted through cannula 100 to contact the tissue. Other devices that may be inserted through cannula 100 to access tissue adjacent distal aperture 116 may include, but are not limited to, one or more devices for removing material, one or more devices for adding material, and one or more devices for cutting, penetrating, puncturing or perforating tissue.

Various measuring devices may be used in conjunction with the cannula of the present invention to monitor physiological parameters such as temperature and pressure. Means for monitoring temperature may include, but are not limited to, thermocouples, thermistors and thermometers. Means for monitoring pressure may include, but are not limited to, pressure transducers and fluid-filled lumens in communication with fluid in a patient's body. Some embodiments of the present invention may comprise some means for monitoring electrical impedance, in order to aid in positioning the cannula within a patient's body.

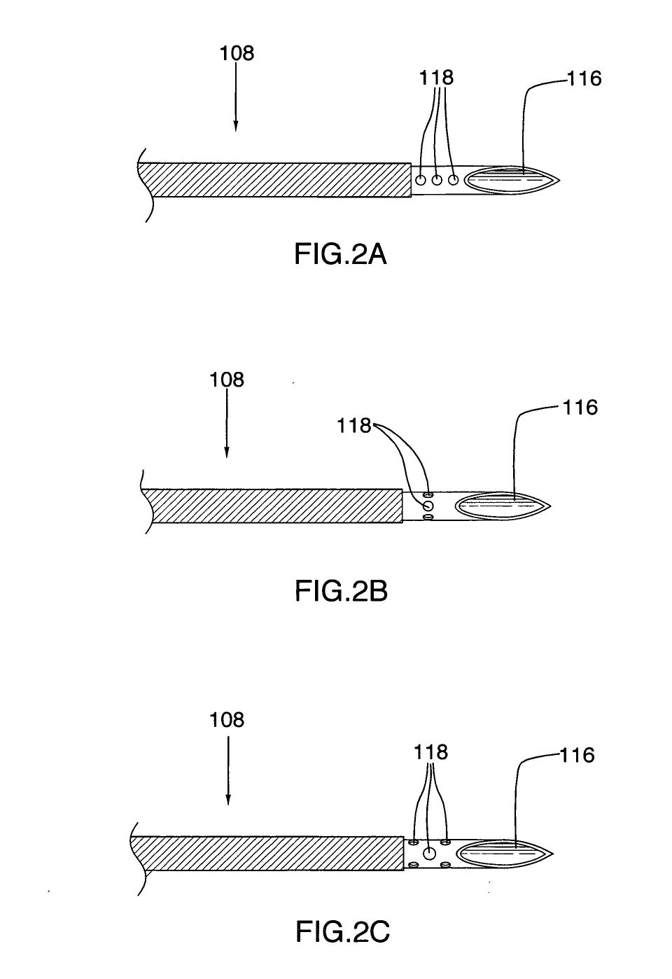

In alternate embodiments, lateral aperture 118 may be disposed on any lateral location of shaft 102, and additional lateral apertures 118 may be present anywhere and in any shape/configuration/arrangement along the surface of shaft 102, as may be useful for a given treatment or tissue. Various embodiments of distal region 114 of cannula 100 are illustrated in FIGS. 2A to 2C. In the embodiment shown in FIG. 2A, cannula 100 comprises a row of lateral apertures 118 parallel to a longitudinal axis of shaft 102. In another embodiment, cannula 100 may comprise numerous lateral apertures 118 in a circumferential arrangement around shaft 102, perpendicular to a longitudinal axis of shaft 102, as shown in FIG. 2B. FIG. 2C depicts an embodiment of cannula 100 with multiple lateral apertures 118 of varying sizes disposed randomly/spaced-apart along cannula 100. In a further embodiment, various apertures may be located at desired locations along, for example, active tip 106 in order to provide regions of electrical discontinuities, where current will not be conducted. Providing such regions may alter the current density along the surface of active tip 106 and may allow for the formation of a lesion with a specified size and/or shape. Although the embodiments illustrated in FIGS. 2A-2C show a cannula with a single lumen, alternate embodiments may comprise a cannula with additional lumens, whereby each lumen may be connected to one or more lateral or distal apertures. In some embodiments, for example, there may be an exclusive, one-to-one relationship between apertures and lumens. In other words, in such embodiments, each aperture may be in communication with a single lumen and there may be one lumen per aperture. In some embodiments, cannula 100 may comprise one or more structures such as, for example, hypotubes or other means for defining a lumen or channel disposed within lumen 110 of shaft 102, each hypotube defining a lumen therethrough. In such embodiments, lateral aperture 118 may be in fluid communication with a first lumen defined by, for example, shaft 102, while distal aperture 116 may be in fluid communication with a second lumen defined by, for example, a hypotube or other structure disposed within shaft 102. Other structures or means for defining a lumen may include, but are not limited to, flexible, rigid or semi-rigid tubing or any other substantially hollow elongate member. As the embodiments of FIGS. 2A and 2C indicate, the number, locations, shapes and configurations of any lateral apertures may vary depending on the specific application or embodiment.

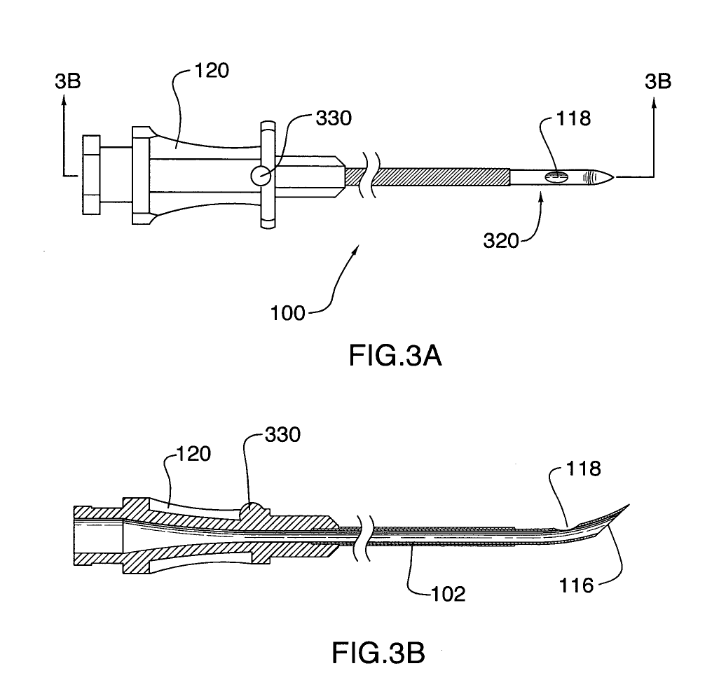

In the embodiment shown in FIGS. 3A and 3B, cannula 100 comprises a curved or rigidly bent distal tip portion 320. With respect to the instant disclosure, the terms ‘bent’ and ‘curved’ are defined to mean having a deviation from a straight line. This may take the form of a rigid bend or a more subtle curve, with various angles or radii of curvature and the curve may be located anywhere along cannula 100. Although the embodiment shown in FIGS. 3A and 3B comprises a bent tip, cannulae with straight tips or varying degrees of curvature may also be used in conjunction with one or more lateral apertures.

In some embodiments, hub 120 may comprise a marker 330 which may be, for example, a visual, tactile, or radiopaque indicator to enable more accurate positioning of cannula 100. Marker 330 may be located, for example, on the same side of cannula 100 as a lateral aperture 118, such that it is aligned with lateral aperture 118, or on the opposite side, thus enabling a user to determine the location and/or orientation of a lateral aperture 118 after cannula 100 has been inserted into a patient's body.

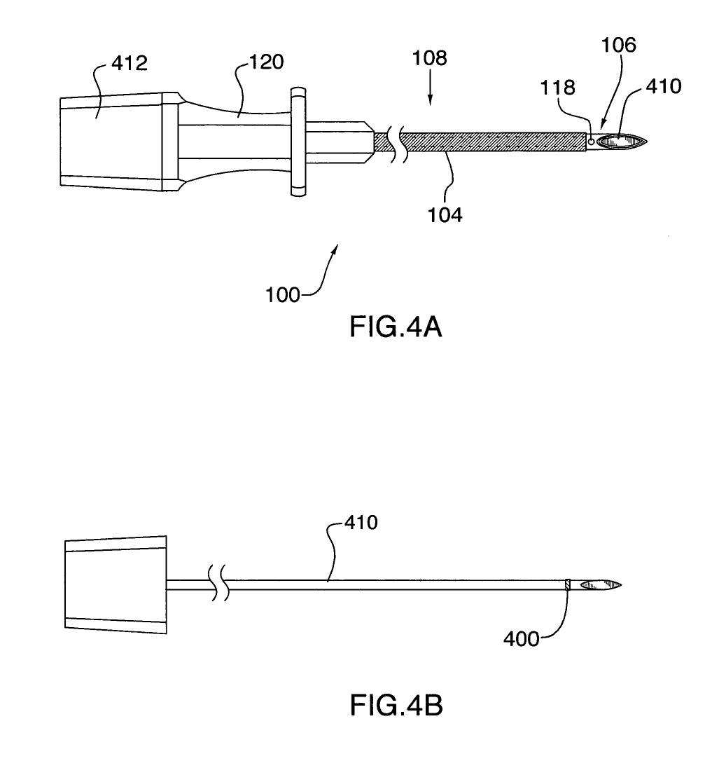

Referring now to FIGS. 4A and 4B, a further embodiment of cannula 100 comprises a lumen 110 sized to hold a stylet 410, which may be removable. Thus, lumen 110 may be capable of receiving at least a portion of stylet 410. In this embodiment, stylet 410, whose position within cannula 100 is shown by a dashed outline, is adapted to facilitate the piercing of a patient's skin and/or tissue by helping to ensure that no tissue is forced into a lumen of the cannula during insertion. Stylet 410 may comprise a cap 412 adapted to cooperatively engage and mate with hub 120 of cannula 100. Stylet 410 may conform to the shape of the distal end of cannula 100; for example, stylet 410 may have a pointed tip with a trocar, conical, bevel, or other shape to allow for easy penetration of tissue when cannula 100 and stylet 410 are introduced into the patient's body. Stylet 410 may be temporarily or permanently attached to cannula 100, for example by welding, soldering, crimping or any other means for bonding. In such embodiments, stylet 410 may not extend the full length of lumen 110 so that lumen 110 can further accommodate a probe. Stylet 410 may fully or partially occlude the distal end of cannula 100. One embodiment of a kit of the present invention may comprise at least one cannula 100, at least one stylet 410 and at least one probe.

In the embodiment shown in FIG. 4B, stylet 410 comprises a marker 400, for example, a radiopaque marker. In this embodiment, radiopaque marker 400 may be used to identify, using fluoroscopic/radiographic imaging for example, a specific portion of the cannula shaft when stylet 410 is fully disposed within a lumen of the cannula. In alternate embodiments, radiopaque marker 400 may be located anywhere on stylet 410 and may adopt any shape or size, may be used in conjunction with one or more markers on cannula 100, or may not be present.

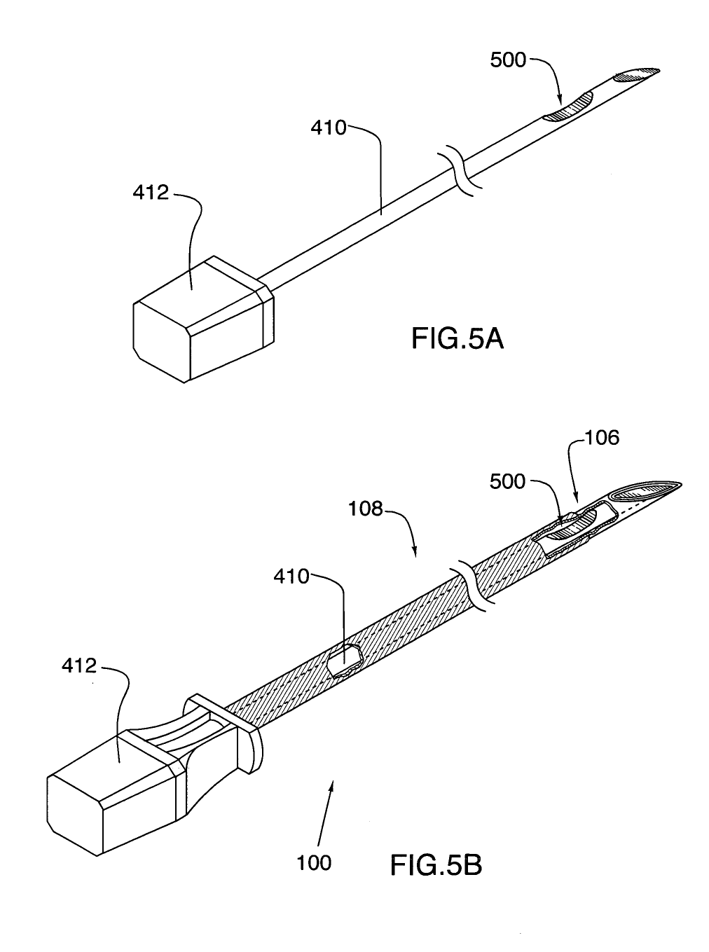

Referring now to the embodiments shown in FIGS. 5A and 5B, the radiopacity of stylet 410 may be reduced at a region 500 by reducing the mass of stylet 410 at that location. The mass of stylet 410 may be reduced by removing material from stylet 410 through grinding techniques or via any other means of removing material. Alternatively, stylet 410 may be originally manufactured with a reduced mass about region 500. In such embodiments, the region 500 of reduced radiopacity of stylet 410 may be aligned with a distal edge of insulated region 108 of cannula 100 and, due to the reduced radiopacity of stylet 410 in this region, the distal edge of insulated region 108 may be more easily distinguished under fluoroscopy, thus allowing a user to determine the precise location of an active tip 106 located adjacent a distal edge of an insulated region 108. In other embodiments, the region of reduced radiopacity of the stylet may be aligned with a lateral aperture 118. Alternatively, these embodiments may be combined such that both active tip 106 and lateral aperture 118 may be simultaneously distinguished under fluoroscopy. In further embodiments, a radiopaque marker may be incorporated onto a stylet 410 having a region 500 of reduced radiopacity, whereby the radiopaque marker may be used to distinguish active tip 106 from insulated region 108, and the region 500 of reduced radiopacity may indicate the location of the lateral aperture. The region 500 of reduced radiopacity may, in some embodiments, be located elsewhere along stylet 410.

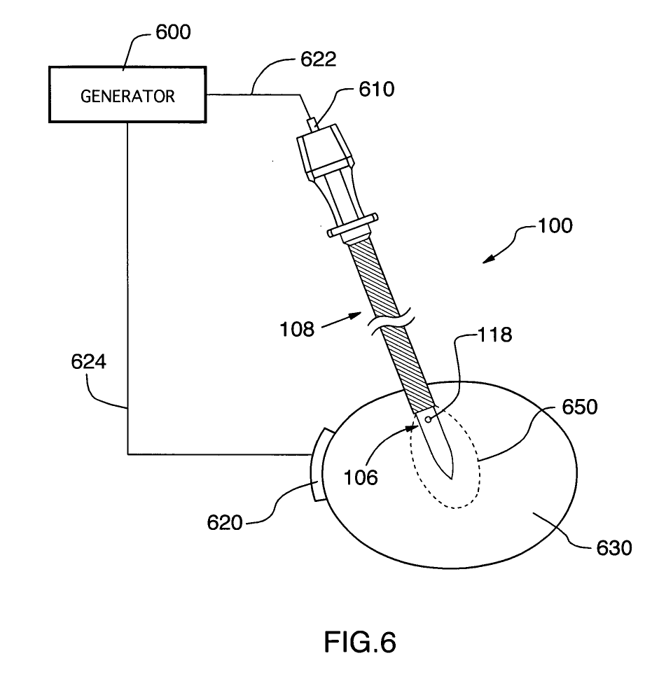

FIG. 6 shows an embodiment of an electrosurgical system incorporating an embodiment of a cannula of the present invention. In this embodiment, the electrosurgical system comprises an energy source 600, a cannula 100 with a lateral aperture 118, a probe 610, a reference electrode 620 and electrical connections 622 and 624. The system may further comprise a stylet 410, a monitoring or measuring device, a means for cooling or various other components. The means for cooling may comprise, for example, peristaltic pumps for supplying a cooling fluid to one or more of cannula 100 and probe 610. In use, and as illustrated in FIG. 6, at least a portion of cannula 100 and probe 610 may be located in a region of a patient's body 630, while reference electrode 620 may be placed at a location on the surface of body 630. The components of the electrosurgical system of FIG. 6 will now be described in greater detail.

Energy source 600 may be any device capable of operating as a source of energy, for example an energy generator. In one embodiment, energy generator 600 is an electrical generator capable of providing high-frequency electrical current. Specifically, energy generator 600 may be operable in a radio-frequency (RF) range, for example, from about 260 kHz to about 1.5 MHz, and may be capable of delivering sufficient power so as to effectively treat a patient's pain. As will be discussed below, treatment of pain may involve the creation of a lesion in a specific neural tissue through heat generated by the application of RF energy. In some embodiments, energy generator 600 may be operable in a range of about 300 kHz to about 600 kHz.

In some embodiments, cannula 100 may be structured so that an electrical connection between probe 610 and cannula 100 is established by the physical apposition of these elements. For example, if cannula 100 is manufactured from a conductive material, probe 610 may be inserted within cannula 100 such that physical contact between cannula 100 and probe 610 may be sufficient to allow for a transfer of electrical energy from the probe to the cannula. In further embodiments, other means for transferring energy from probe 610 to cannula 100 may be utilized. In some embodiments, probe 610 may be cooled, for example, by having one or more internal lumens for the circulation of cooling fluid, or by thermoelectric cooling.

Reference electrode 620 may be sufficiently large to prevent localized heating on the surface of body 630 where reference electrode 620 is placed. In some embodiments, more than one reference electrode may be provided. In alternate embodiments, probe 610 may contain two or more separate electrodes, whereby at least one electrode (the active electrode) may be electrically coupled to cannula 100 and at least one additional electrode may act as a reference electrode. In additional embodiments, reference electrode 620 may be replaced by one or more reference electrodes located on one or more additional probes inserted into the body proximate probe 610. In yet further embodiments, the system may deliver energy in a bipolar configuration, as described below.

Electrical couplings 622 and 624 may be any means for conveying or delivering energy from generator 600 to probe 610 and from reference electrode 620 to generator 600. For example, electrical couplings 622 and 624 may comprise electrical cables/wires along with associated connectors for interfacing with generator 600, probe 610 and reference electrode 620. Various other means for conveying or delivering energy are possible and the invention is not limited in this regard.

In general, electrical current may flow from generator 600 via electrical coupling 622 to probe 610 and via probe 610 to active tip 106. This delivery of energy may result in electrical stimulation or heating of tissue in the region surrounding active tip 106. If the tissue surrounding active tip 106 comprises one or more neural structures, the formation of a lesion 650 may lead to pain relief due to the denervation of said neural structures. Further details of various embodiments of a system of the present invention are described herein below.

Materials/Manufacture

In some embodiments, cannula 100 may be manufactured out of any of a number of conductive materials including but not limited to stainless steel, titanium, a nickel-titanium alloy or other conductive, biocompatible materials able to impart varying degrees of flexibility and strength to cannula 100. In these embodiments, cannula 100 may be overlain with one or more layers of electrically insulating material 104, defining an insulated region 108. Suitable electrically insulating materials 104 may include, but are not limited to, parylene and PTFE. In other embodiments, cannula 100 may be made from a non-conductive material such as, but not limited to PTFE, polyvinylchloride (PVC), or polyurethane, with a conductive material applied overtop of the distal tip to form active tip 106. Cannula 100 may be about 18 to about 22 AWG and about 5 to about 15 cm (approximately 2-4 inches) in length and active tip 106 may be about 2 to about 10 mm (approximately 0.075-0.4 inches) in length. However, cannula 100, as well as the active tip, may be designed in a variety of gauges and lengths and the invention is not limited in this regard.

Method

The present invention also comprises a method of using a cannula with at least one lateral aperture and at least one distal aperture to treat a target treatment site in a body of a patient by delivering energy. Generally speaking, an embodiment of the method may comprise the steps of: inserting a cannula into the body adjacent the target treatment site; delivering energy to the treatment site via the cannula; and delivering a treatment composition to the treatment site via at least one lateral aperture before, after or during the delivery of energy. The delivery of energy may be useful in treating the patient's pain. In one specific embodiment, a more detailed method may proceed as follows: with a patient lying prone, a cannula comprising both a lateral aperture and a distal aperture, and containing a stylet disposed within a lumen of the cannula, is inserted and positioned parallel to the target treatment site to be lesioned using visualization means such as fluoroscopic guidance; a radiopaque or radiolucent marker that may be located on one or more of the cannula and the stylet may provide improved visualization to assist in positioning the cannula. Once positioned, the stylet is removed and replaced by an electrosurgical probe with a protruding distal thermocouple, such that the thermocouple extends in part through the distal aperture of the cannula. Following a test for motor or sensory stimulation, as an added safety measure, an anaesthetic fluid is delivered to the vicinity of the treatment site via one or more lateral apertures. Next, energy is delivered from an energy generator through the probe to the active tip of the cannula in order to create a lesion adjacent the active tip. Energy may be delivered, for example, at a frequency of about 300 kHz to about 600 kHz in order to create a lesion. During energy delivery, the temperature of the tissue adjacent to or in the vicinity of the distal tip of the cannula is monitored by the thermocouple protruding from the electrosurgical probe.

In alternate embodiments, a method of the current invention may comprise a number of variations to the aforementioned embodiment, may omit one or more steps or may add one or more additional steps. The positioning of the patient and the depth and angle of insertion of the cannula may depend on a number of factors, including the location and tissue type at the target treatment site, and on the nature of the procedure(s) to be performed. During and/or following insertion, a number of means for visualizing may be used, including, but not limited to fluorescence, MRI, X-ray, and laparoscopic imaging, and these means for visualizing may or may not be aided by the use of markers such as, but not limited to, radiopaque markers, radiolucent markers, tactile markers, and visual markers, for example, on the proximal portions of the cannula. The step of inserting the cannula into the body may additionally comprise a step of positioning the cannula within the body. This positioning step may involve guiding or steering a part of the cannula using a means for manipulating the cannula, by, for example, actuating a change in the shape of at least a portion of the cannula; twisting, turning, pushing, pulling, expanding, or contracting at least a portion of the cannula; or extending or retracting at least a portion of the cannula. Following insertion, a stylet may be removed, if present, though the method may be performed using cannulae with no stylet or other means for occluding. Alternatively, a stylet may remain within the cannula.

Prior to delivery of energy, one or more additional treatment devices may be inserted and one or more additional treatment procedures may be performed on the tissue. Additional procedures may include, but are not limited to: removal of material, addition of material (including therapeutic fluid agents, such as anaesthetic), and application of cooling. For example, a step comprising the addition of material may comprise the delivery of a treatment composition such as a diagnostic or therapeutic agent including, but not limited to: anesthetic, cooling fluids and chemical, pharmaceutical, and biological agents. Chemical agents may include non-pharmaceutical chemicals, such as ethanol, phenol, chelating agents, tissue sealants, cryogenic fluids, and contrast agents for imaging particular structures of the body, including contrast agents for X-ray, fluoroscopy, ultrasound, computerized tomography (CT), and MRI. Pharmaceutical agents may include drugs commonly available to treat disease, such as pain relievers, anti-cancer agents, antibiotics, anti-thrombotic agents, anti-virals, and enzyme inhibitors. Biological agents may include nucleic acids, amino acids, cells, viruses, prions, biochemicals, vitamins, and hormones. Cooling may be supplied by means other than by delivery of a cooling fluid through an aperture, for example by circulation of a cooling fluid through a closed lumen, or by the use of thermoelectric cooling. In addition, conductive fluids may be delivered to a treatment site in order to allow for the creation of a larger lesion at the treatment site. Alternatively or in addition, delivery of fluid may be used to create a lesion of a desired shape and/or orientation. Material may be added through one or more lateral apertures and/or one or more distal apertures.

The method of the present invention may also comprise, in some embodiments, utilizing the distal aperture in the performance of a secondary procedure. For example, the method may include the insertion of additional treatment devices such that they access the tissue surrounding the cannula through the distal aperture. For example, a device may be inserted through the distal aperture such that at least a portion of the device is in contact with tissue. In some embodiments, the method may comprise the steps of inserting a measuring device into the cannula and using said measuring device to measure a property of the treatment site or of any component of the cannula. Measuring devices may be used to measure, for example, temperature, pressure, or impedance or other physiological parameters. In some embodiments, measuring devices may be operable to directly measure a property of the tissue of the treatment site by contacting said tissue through the distal aperture of the cannula.

The step of inserting a probe into the cannula describes the insertion of any elongated device capable of delivering energy, as described above. Systems used in conjunction with the current method may additionally comprise means for cooling, measurement devices or additional functional elements for performing treatments. In one embodiment, the probe and generator are configured to be operable to deliver stimulation energy to the treatment site, and a measuring device (integral or external to the probe) may be used to measure the response of neural or muscular tissue to said stimulation energy; in this embodiment, the method may comprise the additional steps of delivering energy at a stimulation frequency (for example, about 1 to about 100 Hz) and detecting a response to said energy. Additional functional elements, which may be able to be used to perform secondary procedures may include, but are not limited to elements for removing material from or adding material to the treatment site. Examples of functional elements for removing material include, but are not limited to, clamps, knives, jaws, augers, forceps, scissors, and suction devices. Examples of materials that may be added to a treatment site include, but are not limited to therapeutic agents as described above, sealants, structural or supporting material (synthetic or biological), and materials used to aid in tracing or visualization. Thus, a secondary procedure may involve one or more of adding material to a treatment site and removing material from a treatment site. Cooling means, measuring devices, and additional functional elements may be used prior to, during, or following the step of delivering energy for treatment. Furthermore, in some embodiments, at least a part of the probe may contact the tissue of the treatment site through the distal aperture.

The step of delivering energy may involve delivering energy in a bipolar configuration between two or more cannulae located at spaced apart sites within the body, or delivering energy in a monopolar configuration between one or more cannulae and a reference electrode at a remote location on or in the body. Alternatively, energy may be delivered in various other multipolar or multiphasic configurations. Energy may be delivered continuously, or may be interrupted, for example according to a pre-determined duty cycle. Delivery of energy is an interrupted or discontinuous manner may be referred to as ‘pulsed’ energy delivery.

The step of delivering energy may also be performed, in some embodiments, in conjunction with a step of automatically or manually modifying a treatment procedure (for example, modifying energy delivery) in response to one or more measured properties or parameters. These measured parameters may include, but are not limited to, temperature, position of the probe(s) or impedance. For example, if a temperature measurement is determined to be outside of a desired range, a treatment procedure may be modified by, for example, altering the amount of energy delivered, modifying or modulating a cooling means in some way, or terminating the procedure. In such embodiments, a feedback system may be associated with or incorporated into the energy source so that any modification of a treatment procedure in response to a measured parameter may occur automatically, without any input from a user. Such a feedback system may include, for example, one or more of a processor and a controller. In other embodiments, there may not be an automatic feedback apparatus in place, in which case a user may manually modify a treatment procedure in response to a measured parameter. In addition to modifying a treatment procedure based on measured parameters, this invention also provides for a step of determining the initial parameters to be used in a treatment procedure (for example, the initial maximum power level or tissue temperature, temperature ramp rate, etc.) using information that is known about the particular tissue to be treated. For example, if the tissue to be treated is a patient's sacrum, and if pre-treatment testing reveals specific information about the sacrum (this information may include, but is not limited to: the topology of the sacrum, location of specific nerves, etc.), that information may be used to decide on what parameters to use initially for the treatment procedure.

Embodiments of a treatment procedure as described herein may be useful in order to treat a patient's pain. By creating a lesion in a region of tissue comprising one or more neural structures, the transmission of pain may be blocked. With respect to back pain in particular, such treatment procedures may be applied to several tissues, including intervertebral discs, facet joints, and sacroiliac joints as well as the vertebrae themselves (in a process known as intraosseous denervation). In addition to treating neural structures, the application of RF energy has been effectively used to treat tumors and cardiac tissue, among others.

It should be noted that the terms probe, cannula, stylet etc. are not intended to be limiting and denote any medical and surgical tools that can be used to perform similar functions to those described. In addition, the invention is not limited to be used in the clinical applications disclosed herein, and other medical and surgical procedures wherein a device of the present invention would be useful are included within the scope of the present invention.

The embodiments of the invention described above are intended to be exemplary only. The scope of the invention is therefore intended to be limited solely by the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A cannula comprising:
an elongate shaft comprising a distal region and a proximal region and defining a lumen therebetween and further comprising a wall defining at least one lateral aperture therethrough and a distal end defining at least one distal aperture;
wherein said distal region comprises an electrically exposed and conductive distal tip and wherein an outer surface of said cannula between said distal tip and said proximal region is non-conductive.

2. The cannula of claim 1, wherein said elongate shaft is about 5 cm to about 15 cm in length.

3. The cannula of claim 1, wherein said elongate shaft between said distal tip and said proximal region is made from a non-conductive material.

4. The cannula of claim 1, further comprising an electrically insulating material covering said elongate shaft between said distal tip and said proximal region.

5. The cannula of claim 1, further comprising at least one marker selected from the group consisting of a radiopaque marker, a visual marker and a tactile marker.

6. The cannula of claim 1, further comprising a hub associated with said proximal region, wherein said hub comprises at least one marker.

7. The cannula of claim 6, wherein said at least one marker is aligned with said at least one lateral aperture.

8. The cannula of claim 1, wherein an outer circumferential edge of said at least one lateral aperture is smooth.

9. The cannula of claim 1, wherein at least a portion of said elongate shaft is curved.

10. The cannula of claim 9, wherein at least a portion of said distal tip is curved.

11. The cannula of claim 1, wherein said at least one lateral aperture is in communication with said lumen.

12. The cannula of claim 1, wherein said wall defines more than one lateral aperture therethrough.

13. The cannula of claim 12, wherein each of the lateral apertures is defined by said distal tip.

14. The cannula of claim 12, wherein each of said more than one lateral aperture are defined by said wall in a row parallel to a longitudinal axis of said shaft.

15. The cannula of claim 12, wherein each of said more than one lateral aperture are defined by said wall in a circumferential arrangement perpendicular to a longitudinal axis of said shaft.

16. The cannula of claim 12, wherein said more than one lateral aperture are spaced apart from each other.

17. The cannula of claim 1, further comprising at least one structure defining an additional lumen disposed within said shaft.

18. The cannula of claim 17, wherein said at least one lateral aperture is in communication with one of the lumens and wherein said at least one distal aperture is in communication with another of said lumens.

19. A kit comprising:
at least one cannula comprising an elongate shaft comprising a distal region and a proximal region and defining a lumen therebetween and further comprising a wall defining at least one lateral aperture therethrough and a distal end defining at least one distal aperture;
at least one probe; and
at least one stylet;
wherein said distal region comprises an electrically exposed and conductive distal tip and wherein an outer surface of said cannula between said distal tip and said proximal region is non-conductive.

20. The kit of claim 19, wherein said cannula further comprises a hub sized to cooperatively mate with at least one of said at least one probe and at least one stylet.

21. The kit of claim 19, wherein at least one stylet is attached to said cannula.

22. The kit of claim 19, wherein said lumen is capable of receiving at least a portion of one or more of said at least one probe and said at least one stylet.

23. A electrosurgical system comprising:
at least one cannula comprising an elongate shaft comprising a distal region and a proximal region and defining a lumen therebetween and further comprising a wall defining at least one lateral aperture therethrough and a distal end defining at least one distal aperture, said distal region comprising an electrically exposed and conductive distal tip; and
an energy source for delivering energy to the distal tip;
wherein an outer surface of said cannula between said distal tip and said proximal region is non-conductive.

24. The system of claim 23, further comprising at least one probe operable for connection to said energy source, wherein said lumen is capable of receiving at least a portion of said at least one probe.

25. The system of claim 23, further comprising a means for cooling.

26. The system of claim 23, further comprising at least one reference electrode.

27. The system of claim 23, further comprising at least one measuring device.

28. The system of claim 27, further comprising a feedback system.

29. A method of treating a patient comprising the steps of:
providing a cannula comprising at least one distal aperture and at least one lateral aperture;
delivering a treatment composition to a treatment site via said at least one lateral aperture and said at least one distal aperture; and
delivering energy to said treatment site via said cannula.

30. The method of claim 29, wherein said energy is delivered at a frequency of about 1 Hz to about 100 Hz for stimulating a neural structure.

31. The method of claim 29, wherein said energy is delivered at a frequency of about 300 kHz to about 600 kHz for creating a lesion.

32. The method of claim 30, further comprising a step of delivering energy at a frequency of about 300 kHz to about 600 kHz for creating a lesion.

33. The method of claim 29, wherein said energy is delivered in a monopolar configuration.

34. The method of claim 29, wherein said energy is delivered in a bipolar configuration.

35. The method of claim 29, wherein the delivery of energy is pulsed.

36. The method of claim 29, further comprising a step of measuring a property of at least one of said cannula and said treatment site.

37. The method of claim 36, wherein said property is at least one parameter selected from the group consisting of temperature, pressure and impedance.

38. The method of claim 36, further comprising a step of modifying a treatment procedure in response to the measured property.

39. The method of claim 29, wherein energy is delivered in order to treat pain.

40. The method of claim 29, further comprising a step of performing a secondary procedure utilizing said distal aperture.

41. The method of claim 40, wherein said secondary procedure comprises measuring at least one physiological parameter.

42. The method of claim 40, wherein said secondary procedure comprises introducing a device through said distal aperture.

43. The method of claim 40, wherein said secondary procedure is at least one procedure selected from the group consisting of adding material to a treatment site and removing material from a treatment site.

44. A method of treating a patient comprising the steps of:
providing a cannula comprising at least one distal aperture and at least one lateral aperture;
delivering a treatment composition to said patient via said at least one lateral aperture;
performing a secondary procedure utilizing said at least one distal aperture; and
delivering energy to said patient via said cannula.

45. The method of claim 44, wherein said secondary procedure comprises measuring at least one physiological parameter.

46. The method of claim 44, wherein said secondary procedure comprises introducing a device through said distal aperture.

47. The method of claim 44, wherein said secondary procedure is at least one procedure selected from the group consisting of adding material to a treatment site and removing material from a treatment site.

48. A cannula comprising:
an elongate shaft comprising a distal region and a proximal region and defining a lumen therebetween and further comprising a wall defining at least one lateral aperture therethrough in communication with said lumen and a distal end defining at least one distal aperture in communication with said lumen; and
a hub associated with said proximal region, said hub comprising at least one marker;
wherein said distal region comprises an electrically exposed and conductive distal tip and wherein an outer surface of said cannula between said distal tip and said proximal region is non-conductive and wherein said lateral aperture is defined by said distal tip.
